(19) United States
(12) Patent Application Publication
(10) Pub. No.: US 2026/0167910 A1
(43) Pub. Date: Jun. 18, 2026

(54) VERTICALLY STACKED PHOTOBIOREACTORS WITH ADJUSTABLE REFLECTOR FINS

(71) Applicants: VAAYUNEER SCIENCES PRIVATE LIMITED, Chennai, Tamil Nadu (IN); INDIAN INSTITUTE OF TECHNOLOGY MADRAS (IIT MADRAS), Chennai, Tamil Nadu (IN)

(72) Inventors: Sai SARANGA DAS, Chennai, Tamil Nadu (IN); Suraishkumar G.K., Chennai, Tamil Nadu (IN)

(73) Assignees: VAAYUNEER SCIENCES PRIVATE LIMITED, Chennai, Tamil Nadu (IN); INDIAN INSTITUTE OF TECHNOLOGY MADRAS (IIT MADRAS), Chennai, Tamil Nadu (IN)

(21) Appl. No.: 19/124,842

(22) PCT Filed: Oct. 26, 2023
(86) PCT No.: PCT/IN2023/050981

§ 371 Date: Apr. 25, 2025

(30) Foreign Application Priority Data
Oct. 28, 2022 (IN) 202241061593

(51) Int. Cl. C12M 1/00; C12M 1/06

(52) U.S. Cl. CPC C12M 21/02; C12M 23/22; C12M 23/38; C12M 23/58; C12M 27/02; C12M 27/20; C12M 31/02

(57) ABSTRACT

A photobioreactor system (100) is disclosed. The photobioreactor system (100) comprises a base (102), at least one reflector fin (108) comprising a first end (112) and a second end (114). The first end (112) of the at least one reflector fin (108) is positioned adjacent to the base (102). A stack of photobioreactors (104) is disposed on the base to prepare a culture of photosynthetic organisms. The at least one reflector fin (108) is configured to reflect light rays towards the culture of photosynthetic organisms. The at least one reflector fin (108) is defined with a vertical length at least equal to the elevation of the stack of photobioreactors (104).

### TECHNICAL FIELD

Present disclosure relates to a field of bioprocessing. Particularly, the disclosure pertains to a system for growth and development of photosynthetic organisms and a method for cultivating the photosynthetic organisms.

### BACKGROUND

The information in this section merely provides background information related to the present disclosure and may not constitute prior art(s) for the present disclosure.

Photosynthetic organisms, including cyanobacteria, microalgae, macroalgae, etc. are capable of utilizing light, carbon dioxide (CO₂), and water (H₂O) to produce a wide range of valuable substances such as carbohydrates, proteins, vitamins, lipids, antibiotics, and other bioactive compounds. The photosynthetic organisms function as “cell factories” powered by light, and they have found extensive commercial applications in various industries. The photosynthetic organisms are utilized as animal feed, neutraceutical products as well as for bioremediation, wastewater treatment, and the production of renewable resources like biofuels, bio plastics, bio fertilizers, bio pesticides, and beta carotene.

Photosynthetic microorganisms are primarily cultivated in systems such as open algal ponds or in photobioreactors. Photobioreactors are specifically constructed devices used for culturing photosynthetic organisms, including tissues or cells with photoautotrophic ability. These photobioreactors come in different structural configurations, including but not limited to tubular, flat panel, pouch, thin layer cascade photobioreactors, etc. The cultivation of photosynthetic organisms, particularly microalgae in open ponds, faces numerous disadvantages such as a higher risk of contamination, limited light penetration, and inefficient transfer of gases and liquids. Photobioreactors help mitigate some of these issues by providing better control over culture conditions, such as pH, temperature, or preventing culture contamination. However, the photobioreactors still share certain limitations with open pond systems, which hinder their ability to maximize biomass productivity. These common limitations include the requirement for huge space and the various photosynthetic limitations caused by conditions of excessive or inadequate sunlight in different cases.

In general, both open algal ponds and photobioreactors occupy large landmass spanning over a vast area. The growth rate of photosynthetic organisms, especially microalgae biomass, depends on the intensity of light absorbed by the microalgal cells during the cultivation period. However, as biomass concentration increases, the availability of light for the cells decreases due to shading caused by microbial growth. Consequently, microalgal biomass tends to receive sunlight only up to a certain depth from the surface where sufficient light can penetrate from the surface of the culture medium. Light beyond this depth is limited due to the self-shading effects of microbial growth and the attenuation of light in water. In the case of, say, tubular photobioreactors, there needs to be adequate spacing between successive rows of tubular photobioreactors (a row of tubular photobioreactors is defined as a set of tubular photobioreactors placed adjacent to each other with a spacing between them) to prevent the shading of one row of tubular photobioreactor by another. As a result, cultivating photosynthetic organisms requires large areas of land, contributing to the costly nature of these cultivation practices.

The present disclosure aims to overcome these limitations associated with open pond systems and photobioreactors by enabling increased biomass productivity in smaller areas and in a more cost-effective manner.

### SUMMARY

One or more shortcomings of the prior art are overcome by the system/assembly as claimed, and additional advantages are provided through the provision of the system/assembly/method as claimed in the present disclosure. Additional features and advantages are realized through the techniques of the present disclosure. Other embodiments and aspects of the disclosure are described in detail herein and are considered a part of the claimed disclosure.

In one non-limiting embodiment of the present disclosure, a photobioreactor system is disclosed. The photobioreactor system comprises a base, at least one reflector fin comprising a first end and a second end. The first end of the at least one reflector fin is positioned adjacent to the base. A stack of photobioreactors is disposed on the base to prepare culture of photosynthetic organisms. The at least one reflective fin is configured to reflect light rays towards the culture of photosynthetic organisms. The at least one reflector fin is defined with a vertical length at least equal to the elevation of the stack of photobioreactors.

In an embodiment of the present disclosure, the individual photobioreactors comprising the stack of photobioreactors are positioned one above the other.

In an embodiment of the present disclosure, the second end of the at least one reflector fin is adjustable with respect to the base.

In an embodiment of the present disclosure, the at least one reflector fin is inclined at an angle (α) from the base.

In an embodiment of the present disclosure, the length of the at least one reflector fin is such that even after adjusting for the angle (α) from the base, the at least one reflector fin has a slant height at least equal to the elevation of the stack of photobioreactors.

In an embodiment of the present disclosure, a gap (G) separates two adjacent reflector fins.

In an embodiment of the present disclosure, the at least one reflector fin is coated with a reflective material.

In an embodiment of the present disclosure, the gap (G) separating each of the reflector fins from another is covered by a material.

In an embodiment of the present disclosure, the first end of the at least one reflector fin is positioned at a distance “x” from the base.

In an embodiment of the present disclosure, the stack of photobioreactors comprises a culture circulation system having at least one baffle and one impeller configured to circulate essential nutrients to photosynthetic organisms.

In an embodiment of the present disclosure, the stack of photobioreactors comprises at least one pump configured to circulate essential nutrients to photosynthetic organisms.

In an embodiment of the present disclosure, the photobioreactor system comprises a translucent or an opaque cover configured to cover a top surface of the stack of photobioreactors.

In an embodiment of the present disclosure, the cover is positioned at a distance from the top surface of the stack of photobioreactors and the cover defines a plurality of apertures configured to allow entry of light rays through the cover.

In an embodiment of the present disclosure, the apertures present in the cover can be opened and closed dynamically using an actuator.

It is to be understood that the aspects and embodiments of the disclosure described above may be used in any combination with each other. Several of the aspects and embodiments may be combined together to form a further embodiment of the disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF FIGURES

The novel features and characteristics of the disclosure are set forth in the description. The disclosure itself, however, as well as a preferred mode of use, further objectives, and advantages thereof, will best be understood by reference to the following description of an illustrative embodiment when read in conjunction with the accompanying drawings. One or more embodiments are now described, by way of example only, with reference to the accompanying drawings wherein like reference numerals represent like elements and in which:

FIG. 1 illustrates a schematic view of a vertically stacked photobioreactor system with adjustable reflector fins, wherein each photobioreactor in the stack is a flat panel photobioreactor, in accordance with an embodiment of the present disclosure.

FIG. 2 illustrates a schematic view of a plurality of stacked photobioreactor systems with adjustable reflector fins, in accordance with an embodiment of the present disclosure.

FIG. 3 illustrates a graph corresponding to the effect of light intensity as a function of reflector fin distance, in accordance with an embodiment of the present disclosure.

FIG. 4 illustrates a schematic view of a tubular photobioreactor, in accordance with an embodiment of the present disclosure.

FIG. 5 illustrates a schematic view of the tubular photobioreactor, in accordance with another embodiment of the present disclosure.

FIG. 6 illustrates a top-down perspective view of the photobioreactor system when tubular bioreactors are used, in accordance with an embodiment of the present disclosure.

FIG. 7 illustrates a graph corresponding to the effect of light intensity as a function of the number of reflector fins present in the system, in accordance with an embodiment of the present disclosure.

Skilled artisans will appreciate that elements in the drawings are illustrated for simplicity and have not necessarily been drawn to scale. For example, the dimensions of some of the elements in the drawings may be exaggerated relative to other elements to help to improve understanding of embodiments of the present disclosure.

### DETAILED DESCRIPTION

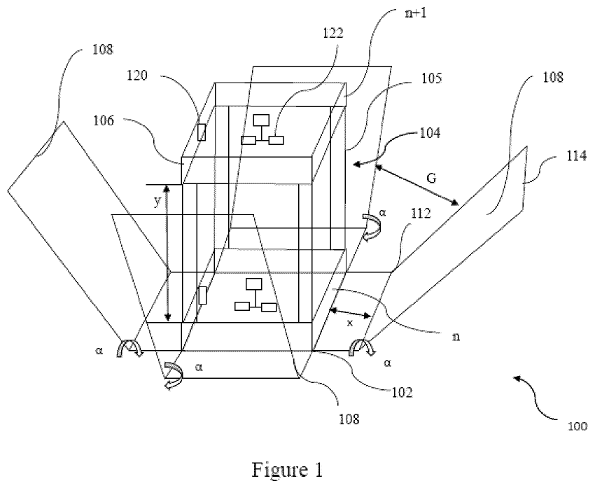

While the disclosure is susceptible to various modifications and alternative forms, specific embodiments thereof have been shown by way of example in the FIGS. 1 to 7 and will be described in detail below. It should be understood, however that it is not intended to limit the disclosure to the particular forms disclosed, but on the contrary, the disclosure is to cover all modifications, equivalents and alternatives falling within the scope of the disclosure as defined by the appended claims.

In the present disclosure, the term “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiments thereof have been shown by way of example in the drawings and will be described below. It should be understood, however, that it is not intended to limit the disclosure to the particular forms disclosed, but on the contrary, the disclosure is to cover all modifications, equivalents, and alternatives falling within the scope of the disclosure.

The terms “comprises”, “comprising”, or any other variations thereof, are intended to cover a non-exclusive inclusion, such that the product that comprises a list of components does not include only those components but may include other components not expressly listed or inherent to such device/system. In other words, one or more elements in the product proceeded by “comprises . . . a” does not, without more constraints, preclude the existence of other elements or additional elements in the devices.

Henceforth, the present disclosure is explained with the help of one or more figures of exemplary embodiments. However, such exemplary embodiments should not be construed as limitations of the present disclosure. The terms reactors, photobioreactors and bioreactors are interchangeably used.

Referring to FIG. 1, a photobioreactor system (100) is disclosed to create an optimal environment for the growth of photosynthetic organisms. The photobioreactor system (100) comprises a base (102) configured to provide stability and support to the photobioreactor system (100). Furthermore, the photobioreactor system (100) comprises at least one reflector fin (108) comprising a first end (112) and a second end (114). The at least one reflector fin (108) is configured to reflect light rays towards the culture of photosynthetic organisms. The first end (112) of the at least one reflector fin (108) is positioned adjacent to the base (102). In an embodiment, the first end (112) of the at least one reflector fin (108) is positioned at a distance “x” from the base (102). Further, the second end (114) of the at least one reflector fin (108) is adjustable with respect to the base (102). In an embodiment, the distance “x” is in the range of 1 mm to 50 m. In an embodiment, the distance between two successive stages of a photobioreactor (n, n+1) is “y”. In an embodiment, the distance “y” is in the range of 1 cm to 1 m. In an embodiment, the at least one reflector fin (108) is coated with a reflective material. In an embodiment, the at least one reflector fin (108) are coated with materials that have a high reflectance comprising but not limited to aluminium, silver etc.

In an embodiment, the base (102) is constructed for the positioning of a stack of photobioreactors (104). Each photobioreactor (106) within the stack of photobioreactors (104) is configured to prepare the culture of photosynthetic organisms. In an embodiment, the bioreactor (106) can be of various geometries, including but not limited to flat panel photobioreactors, tubular photobioreactors, thin layer cascade photobioreactors, etc. without limiting the scope of the disclosure. In an embodiment, each of the photobioreactor (n) of the stack of photobioreactors (104) is independent or may be interconnected and is separated from the successively stacked photobioreactor (n+1). In an embodiment, the successively stacked photobioreactors are positioned one above the other by a specific height “y”. The successive photobioreactors (n+1) are stacked vertically and are supported by means of one or more columns (105). In an embodiment, each of the at least one reflector fin (108) is at a tilt angle (α) with respect to the base (102). In an embodiment, each of the at least one reflector fin (108) is separated from an adjacent reflector fin (108) through a gap (G). In an embodiment, the gap (G) separating each of the reflector fins (108) from another is covered by a material that is either reflective or non-reflective depending on the intended purpose or configuration requirement of the material used to cover the gap (G). This flexibility in the configuration allows for the use of suitable material as per the requirement of the photobioreactor system (100). In an embodiment, the photobioreactor system (100) comprises four reflector fins (108), each positioned at the side of the stack of photobioreactors (104). The four sides of the photobioreactor system (100) are enclosed by the reflector fins (108) in a manner that allows light to pass through the gaps (G) between the reflector fins (108) as well as through the top of the photobioreactor system (100). These configurations enable precise regulation of light exposure within the photobioreactor system (100). In an embodiment, light entering through the top of the photobioreactor system (100) is limited by a cover (not shown in the figures) over the stack of photobioreactors (104) placed at a distance from the top surface of the stack of photobioreactors (104). In this configuration, light enters from the top through the apertures that are present in the cover (not shown in the figures)

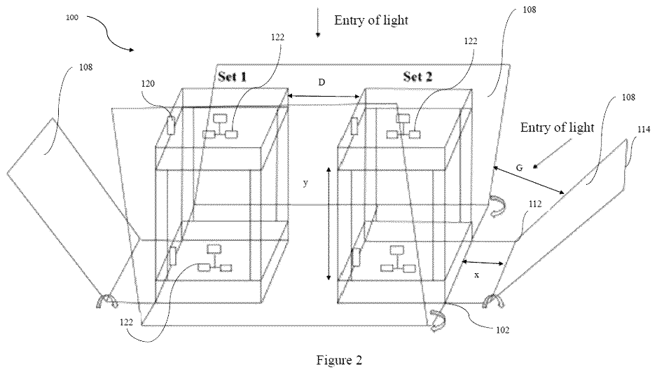

The at least one reflector fin (108) is defined with a vertical length at least equal to the elevation of the stack of photobioreactors (104). In an embodiment, the vertical length of the at least one reflector fin (108) on all four sides of the photobioreactor system (100) should be at least equal to the elevation of the entire reactor setup of the stack of photobioreactors (104). Alternatively, the vertical length of the at least one reflector fin (108) should match the elevation of the largest bioreactor stage of the stack of photobioreactors (104) if there are variations in the size of the bioreactors. This height alignment is crucial to ensure optimal light distribution over a substantial part of the photobioreactor system (100). In an embodiment, the breadth of the reflector fins (108) is configured to entirely cover the breadth of the stack of photobioreactors (104). In addition to ensuring an optimal light distribution over a substantial part of the photobioreactor system (100), this comprehensive coverage is also essential to prevent any substantial part of the stack of photobioreactors (104) from being exposed directly to the full intensity of sunlight. Instead, light should only enter from the top or pass through any potential gaps (G) between the reflector fins (108). Another advantage that this type of setup confers is that due to the optimal light distribution environment that is provided due to the presence of reflector fins (108), the spacing between successive stacks of photobioreactors (104) like flat panel stacks (the distance ‘D’ as shown in FIG. 2) or stacks of tubular photobioreactor rows can be reduced. This has the effect of increasing the areal productivity of these systems.

In an embodiment, the at least one reflector fin (108) is inclined at an angle (α) from the base (102). The vertical length of the at least one reflector fin (108) is such that even after tilting the reflector fin (108) at an angle (α) from the base (102), the at least one reflector fin (108) has a slant height at least equal to the elevation of the stack of photobioreactors (104). In an embodiment, the angle (α) of at least one reflector fin (108) ranges from 10° to 150° from the base (102) of the photobioreactor system (100). However, these ranges should not be considered as limitations on the tilt angle (α) of the at least one reflector fin (108). In an embodiment, the actuation of the reflector fins (108) to a specific tilt angle position is carried out by an actuator (not shown in the figures) so that the reflector fins (108) displace from a first position to a second position. The actuator may be connectable to a control unit (not shown in the figures) which sends out control signals to the actuator for actuation of the reflector fins (108). In an embodiment, the actuator may be powered, for example, the actuator may be electronically controlled through a control means which is in turn connected to a control unit in order to displace the reflector fin (108) from a first position to a second position to the required tilt angle.

In an embodiment, the stack of photobioreactors (104) comprises a culture circulation system having at least one baffle (120) and one impeller (122) configured to circulate essential nutrients to photosynthetic organisms.

In another embodiment, the culture circulation system comprises at least one pump (not shown in the figure) configured to circulate essential nutrients to photosynthetic organisms.

Referring to FIG. 2, in one embodiment, a plurality of stacks of photobioreactors (104) are placed next to each other at a distance ‘D’ between each other. In an embodiment, the distance ‘D between the plurality of stacks of photobioreactors (104) can be modulated dynamically using actuators (not shown in the figures). In an embodiment, the length of the photobioreactor system (100) is in the range of 10 cm to 2 km, breadth in the range of 10 cm to 2 km and height in the range of 10 cm to 1 km. However, these ranges should not be considered as limitations and any dimensional photobioreactor system (100) may be considered based on requirement.

In an embodiment, the upper surface of at least one flat panel bioreactor (106) is configured to be opaque. This characteristic is applied in conjunction with any existing cover that may be in place across the setup of the stack of photobioreactors (104). Alternatively, in another embodiment, the upper surface of at least one flat panel bioreactor (106) is configured to be translucent. This is also combined with the presence of any cover that may exist across the setup of the stack of photobioreactors (104).

In an embodiment, the circular lid (118) of at least one tubular photobioreactor (106) is configured to be opaque. This characteristic is applied in conjunction with any existing cover that may be in place across the setup of the stack of photobioreactors (104). Alternatively, in another embodiment, the circular lid (118) of at least one tubular photobioreactor (106) is configured to be translucent. This is also combined with the presence of any cover that may exist across the setup of the stack of photobioreactors (104).

### Experimental Results

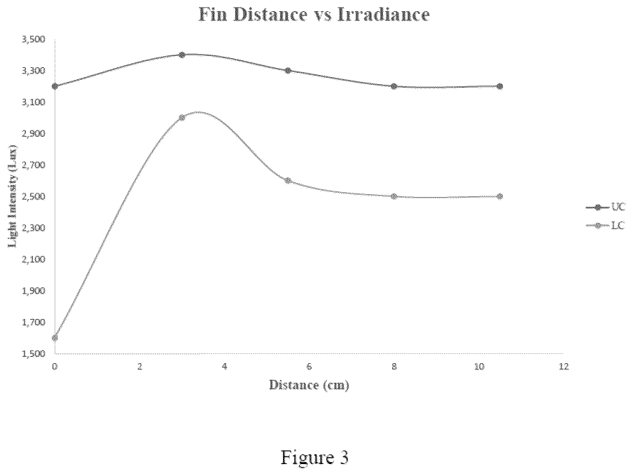

Example 1: Referring to FIGS. 1 and 3, the impact of the distance “x” between the at least one reflector fin (108) and the base (102) of a two-stage photobioreactor system (100) on changes in light intensity is studied. In an embodiment, the photobioreactor system (100) comprises of four reflector fins (108) on four sides of the photobioreactor system (100). The height, breadth and thickness of the reflector fins (108) were 24 cm, 16 cm and 0.3 cm respectively. The tilt angle (α) of these fins was adjusted using a hinge and rope-based mechanism. Ropes of diameter 3 mm were fastened to the back of each of the reflector fins (108) through a provision (not shown in figure). The other end of the rope was fastened to the upper chamber (n+1) in the provisions that were provided on each side of the upper chamber (not shown in figure). This mechanism held the individual reflector fins (108) at a particular desired tilt angle (α). Both the chambers contained baffles (120) that are attached at the centre of the four sides of the upper (n+1) and lower (n) chambers. The height, breadth and thickness of each baffle (120) were 5 cm, 1.2 cm and 0.3 cm respectively. The two-stage photobioreactor system (100) comprises an upper chamber (UC) and a lower chamber (LC). The thickness of the acrylic material from which the upper and lower chambers were constructed was 0.5 cm. The vertical spacing between the two chambers was 9.3 cm. The length, breadth and the height (excluding the thickness of the acrylic material) of the upper and lower chambers were 15 cm, 15 cm and 5 cm respectively. For an experimental study, the tilt angle (α) of the all the reflector fins (108) was adjusted to 90°. A CFL light source (not shown in figure) was placed parallel to the plane of the base (102) of the photobioreactor system (100). The CFL light source was positioned at the top of the photobioreactor system (100). The length of the CFL light source was less than the length of the photobioreactor system (100). The initial distance between the four vertical reflector fins (108) and the stack of the photobioreactors (104) was 3 cm. The vertical distance between the light source and the upper chamber (UC) was adjusted such that the average light intensity (defined as the average of light intensities incident on the four corners of a given chamber) of the upper chamber (UC) as measured by a HTC LX-101A light meter was in the range of 3,200 lux. Post this, the distance between the stack of photobioreactors (104) and all four reflector fins (108) was changed uniformly and the average light intensity was measured. The experimental results for both the upper chamber (UC) and the lower chambers (LC) are presented in a graph shown in FIG. 3. The angle (α) was maintained at 90° at all distances. The intensity measurement represents the average light intensity of the upper chamber (UC) and the lower chamber (LC). The standard deviation of the light intensity at any corner of any given chamber did not exceed 20% of the average light intensity of the respective chamber.

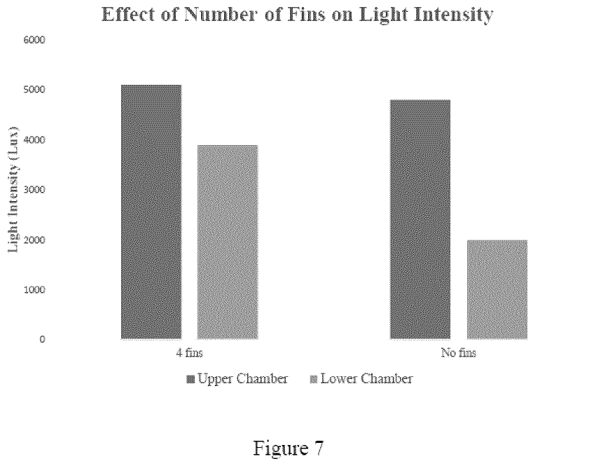

Example 2: In an embodiment, the photobioreactor system (100) comprises of four reflector fins (108) on four sides of the photobioreactor system (100). Referring to FIGS. 1 and 7, the height, breadth and thickness of the reflector fins (108) were 24 cm, 16 cm and 0.3 cm respectively. The tilt angle (α) of these fins were adjusted using a hinge and rope-based mechanism. Both chambers contained baffles (120) that are attached at the centre of the four sides of the upper (n+1) and lower (n) chambers. The height, breadth and thickness of each baffle (120) were 5 cm, 1.2 cm and 0.3 cm respectively. The two-stage photobioreactor system (100) comprises an upper chamber (UC) and a lower chamber (LC). The thickness of the acrylic material from which the upper and lower chambers were constructed was 0.5 cm. The vertical spacing between the two chambers was 9.3 cm. The length, breadth and height (excluding the thickness of the acrylic material) of the upper and lower chambers were 15 cm, 15 cm and 5 cm respectively. For an experimental study, the tilt angle (α) of all the reflector fins (108) was adjusted to 90°. A CFL light source (not shown in figure) was placed parallel to the plane of the base (102) of the photobioreactor system (100). The length of the CFL light source was less than the length of the photobioreactor system (100). The distance between each of the four reflector fins (108) and the stack of photobioreactors (104) was 3 cm. The light intensity at the center of the upper and lower chambers as measured by a HTC LX-101A light meter were in the range of 5,100 lux and 3,900 lux respectively. Post this, all the four reflector fins (108) were removed and the photobioreactor system (100) was placed at the same position with respect to the CFL light source and the light intensity measured. This time, the light intensity in the upper and lower chambers were in the range of 4,800 lux and 2,000 lux respectively. It is pertinent to note that by the removal of the reflector fins (108), the light at the center of the lower chamber falls much more significantly than that in the upper chamber (5.9% and 48.7% drop in the light intensities in the upper and lower chambers respectively).

Example 3: In an embodiment, the photobioreactor system (100) comprises four reflector fins (108) on four sides of the photobioreactor system (100). The base (102) of the system (100) was a square of side 15 cm. The base (102) had a circular elevation of a diameter of 10 cm and a thickness of 0.3 cm. Each reflector fin (108) measured 30 cm in height, 0.5 cm in thickness, and 10 cm in breadth. These reflector fins (108), inclined at an angle of 98° with respect to the base (102) were connected to it via a hinge based mechanism. In this configuration, each of the top tip of each of the adjacent reflector fins (108) was in touch with each other. A CFL light source of power 18 W was placed at a plane perpendicular to the plane of the base (102). The distance between the ends of the CFL lamp and the reflector fins (108) on all four sides was 3 cm. With four reflector fins (108), the light intensity as measured by a HTC LX-101A light meter at the centre of the base (102) was in the range of 3,500 lux. After this, two reflector fins (108) facing the north and south sides of the setup were removed. With two reflector fins (108), the east and the west side of the reflector fins (108) were maintained at an angle of 98° with respect to the base (102) using a hinge and rope-based mechanism. In this configuration, the light intensity was in the range of 1,350 lux. Thus, for a given light source at a given intensity, the configuration of four reflector fins (108) on all four sides of the photobioreactor system (100) captures a higher quantum of light per unit area than that configuration in which only two reflector fins (108) were used.

Examples 1, 2 and 3 are significant since light is one of the major factors that control the growth rate of a photosynthetic organism. It is also pertinent to note that different strains and species of photosynthetic organisms like microalgae, macroalgae, various plant species, etc. have different light intensities for optimal growth. Therefore, by modulating the quantum of light incident per unit area of the space enclosed by the reflector fins (108) by changing the variables such as the tilt angle (α), the spacing between the reflector fins (108) and the stack of photobioreactors (104), etc. a person skilled in the art can design appropriate systems that are suitable for the optimal growth of various photosynthetic organisms depending upon their optimal light intensity.

Example 4: In an embodiment, the photobioreactor system (100) comprises of four reflector fins (108) on four sides of the photobioreactor system (100). Referring to FIG. 1, a photosynthetic organism, for example, a Chlorella species was grown in a two-stage flat panel photobioreactor using ASNIII media. In one such experiment, the height, breadth and thickness of the reflector fins (108) were 24 cm, 16 cm and 0.3 cm respectively. The tilt angle (α) of these reflector fins (108) was adjusted using a hinge and rope-based mechanism. Both chambers contained baffles (120) that are attached at the centre of the four sides of the upper (n+1) and lower (n) chambers. The height, breadth and thickness of each baffle (120) were 5 cm, 1.2 cm and 0.3 cm respectively. The thickness of the acrylic material from which the upper and lower chambers were constructed was 0.5 cm. The vertical spacing between the two chambers was 9.3 cm. The length, breadth and height (excluding the thickness of the acrylic material) of the upper and lower chambers were 15 cm, 15 cm and 5 cm respectively. Two lids in the shape of a square of side 16 cm with a provision for fitting a motor at the centre were used to close the two chambers. A 12V motor (not shown in the figure) was placed in the provision. The shaft of the motor was connected to a two-blade impeller (122). The length of each blade is 3.75 cm. The total length of the impeller (122) from one side to the other is 10 cm. Each blade of the impeller was machined on one side so that it can be attached to an elliptical provision whose length of the major axis is 2.5 cm and has a thickness of 0.3 cm. Each blade was attached to the opposite ends of the elliptical provision that was then attached to the impeller shaft. The height and the diameter of the impeller shaft were 1.8 cm and 1 cm respectively. When attached to the shaft of the motor, the distance between the impeller (122) and the bottom face of the upper and lower chambers (excluding the thickness of the acrylic) was 2 cm. Both the impellers (122) in the upper and lower chambers were rotated at 90 rpm. The distance between the reflector fins (108) and the stack of photobioreactors (104) on all four sides was 3 cm. Before the addition of the culture, the tilt angle (α) of all four reflector fins (108) was adjusted such that the average intensity of light of the upper and lower chambers as measured by a HTC LX-101A light meter were in the range of 4,600 lux and 3,900 lux respectively. The standard deviation of the light intensity at any corner of any given chamber did not exceed 20% of the average light intensity of the respective chambers. A light regime with a light:dark cycle of 12 hours was maintained (light cycle: 12:00 pm to 12:00 am; dark cycle: 12:00 am to 12:00 pm). The motor in the upper and the lower chambers were switched off periodically in the following manner: from 2:00 am to 2:15 am; 6:15 am to 6:30 am; 10:30 am to 10:45 am; 2:45 pm to 3:00 pm and from 7:00 pm to 7:15 pm. The initial OD₇₃₀ of the culture was 0.26. The volume of culture in both the upper and the lower chambers was 600 ml each. During the course of the experiment, the pH of the culture was in the range of 7.0-7.5. The final OD after 3 days in the upper and lower chambers was 0.68 and 0.50 respectively.

Example 5: In an embodiment, the photobioreactor system (100) comprises of four reflector fins (108) on four sides of the photobioreactor system (100). Referring to FIG. 1, a photosynthetic organism, for example, a Chlorella species was grown in a two-stage flat panel photobioreactor using ASNIII media. The Chlorella species used was the same as that used in example 4. The system (100) used in this experiment was similar to that used in example 4 except for the fact that there were no reflector fins (108) present in the system (100). In one such experiment, both the chambers contained baffles (120) that were attached at the centre of the four sides of the upper (n+1) and lower (n) chambers. The height, breadth and thickness of each baffle (120) were 5 cm, 1.2 cm and 0.3 cm respectively. The thickness of the acrylic material from which the upper and lower chambers were constructed was 0.5 cm. The vertical spacing between the two chambers was 9.3 cm. The length, breadth and height (excluding the thickness of the acrylic material) of the upper and lower chambers were 15 cm, 15 cm and 5 cm respectively. Two lids in the shape of a square of side 16 cm with a provision for fitting a motor at the centre were used to close the two chambers. A 12V motor (not shown in the figure) was placed in the provision. The shaft of the motor was connected to a two-blade impeller (122). The length of each blade is 3.75 cm. The total length of the impeller (122) from one side to the other is 10 cm. Each blade of the impeller was machined on one side so that it can be attached to an elliptical provision whose length of the major axis is 2.5 cm and has a thickness of 0.3 cm. Each blade was attached to the opposite ends of the elliptical provision that was then attached to the impeller shaft. The height and the diameter of the impeller shaft are 1.8 cm and 1 cm respectively. When attached to the shaft of the motor, the distance between the impeller (122) and the bottom face of the upper and the lower chambers (excluding the thickness of the acrylic) was 2 cm. Both the impellers (122) in the upper and lower chambers were rotated at 90 rpm. Before the addition of culture, the average intensity of light in the upper and the lower chambers as measured by a HTC LX-101A light meter were in the range of 4,600 lux and 1,950 lux respectively. The standard deviation of the light intensity at any corner of any given chamber did not exceed 20% of the average light intensity of the respective chamber. A light regime with a light:dark cycle of 12 hours was maintained (light cycle: 12:00 pm to 12:00 am; dark cycle: 12:00 am to 12:00 pm). The motor in the upper and the lower chambers were switched off periodically in the following manner: from 2:00 am to 2:15 am; 6:15 am to 6:30 am; 10:30 am to 10:45 am; 2:45 pm to 3:00 pm and from 7:00 pm to 7:15 pm. The initial OD₇₃₀ of the culture was 0.25. The volume of culture in both the upper and the lower chambers was 600 ml each. During the course of the experiment, the pH of the culture was in the range of 7.0-7.5. The final OD after 3 days in the upper and lower chambers was 0.61 and 0.36 respectively.

Examples 4 and 5 show the significance of using reflector fins (108) on the growth of a photosynthetic organism. In these instances, the increase in productivity was 11.5% and 39% for the upper and the lower chambers respectively when there were reflector fins (108) on all four sides of the stack of photobioreactors (104) (example 4) compared to the scenario where no reflector fins (108) were present (example 5).

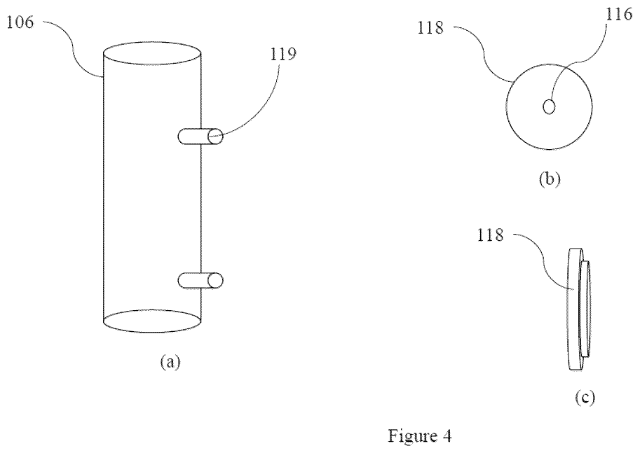

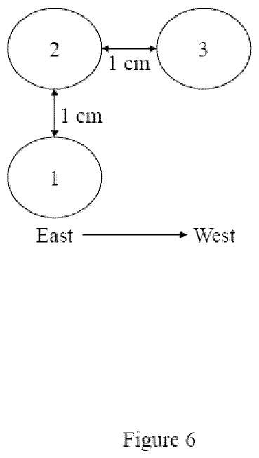

Example 6: Referring to FIG. 4, the tubular photobioreactor (106) has the following dimensions: the inner diameter is 3.5 cm, and the outer diameter is 4 cm, with a height of 20 cm. The bottom of the photobioreactor is 0.3 cm thick. The tubular photobioreactor (106) is sealed by a two-layered circular cap (118), as shown in FIGS. 4 (b) and 4(c) showing the top and the side view of the cap respectively. The upper layer of the cap has a diameter of 4 cm, while the lower layer's diameter is 3.4 cm, and both layers are 0.3 cm thick. There is a 0.5 cm diameter hole (116) in the centre of the cap (118). The reactor (106) has two outlets (119) on the same side positioned along the longitudinal axis of the bioreactor. The upper outlet is positioned 5 cm from the top of the photobioreactor (106), while the lower outlet is located 1 cm from the bottom of the photobioreactor (106). Both outlets (119) have inner and outer diameters of 0.5 cm and 0.8 cm respectively. To connect these outlets (119) to a pump, a silicon tube (not shown in the figure) with inner and outer diameters of 0.5 cm and 0.8 cm, respectively, was used. This tube had a length of 14 cm for the upper outlet and 3 cm for the lower outlet. It was connected to a polyurethane tube with inner and outer diameters of 0.4 cm and 0.6 cm, respectively, and a length of 3 cm. This connection allowed for the upper and lower outlets to be connected to the inlet and outlet of a 12V Kamoer pump (model EDLP600-D12A) respectively, which has a flow rate of 600 ml/min. A photosynthetic organism, for example a Chlorella species was grown in three tubular photobioreactors positioned on a square platform of side 15 cm surrounded by reflector fins (108) on four sides. The platform had a circular elevation of a diameter of 10 cm and a thickness of 0.3 cm. Each reflector fin (108) measured 30 cm in height, 0.5 cm in thickness, and 10 cm in breadth. These reflector fins (108), inclined at an angle of 98° with respect to the base (102) were connected to the platform via a hinge-based mechanism. In this configuration, each of the top tip of each of the adjacent reflector fins (108) was in touch with each other. In one such experiment, after placing the four reflector fins (108) and before placing the tubular photobioreactors (106), the light intensity at the centre of the base (102) as measured by a HTC LX-101A light meter was in the range of 3,500 lux. Sodium bicarbonate, at a daily dosage of 1 mg, was introduced to the culture. The reactor's lid (118) was both opaque and punctured at its centre to allow bicarbonate injection. The spacing between the outer edges of the three tubular photobioreactors (106) is shown in FIG. 6. In this configuration, the distance between the outer edges of the tubular bioreactors (106) and the first end of the reflector fin (112) was 3 cm. The silicon tubes connecting the reactor (106) to the pump traversed through the gaps between the nearest two reflector fins (108). A light:dark cycle of 12 hours: 12 hours was maintained (light cycle: 12:00 pm to 12:00 am; dark cycle: 12:00 am to 12:00 pm). The reactor (106) was switched off periodically in the following manner: from 2:00 am to 2:15 am; 6:15 am to 6:30 am; 10:30 am to 10:45 am; 2:45 pm to 3:00 pm and from 7:00 pm to 7:15 pm. During the course of the experiment, the pH of the culture was in the range of 7.0-7.5. With an initial OD₇₃₀ of 0.25, the culture demonstrated varying growth rates in the three bioreactors (1, 2 and 3 as referred to in FIG. 6), resulting in final OD₇₃₀ values of 0.49, 0.47, and 0.61. The total duration of the experiment was 10 days. The specific growth rates for the three tubular photobioreactors (106) were 0.067, 0.063 and 0.088 per day.

Referring to FIG. 6, as an example, when viewing a tubular photobioreactor system (100) from a top-down perspective view, the process of numbering the photobioreactors (106) involves assigning sequential numbers or labels to different sections or units of the photobioreactor (106) as you move from the east side to the west side. This numbering system helps identify and locate specific components or areas within the photobioreactor system (100), making it easier to reference or work with different parts of the equipment or system.

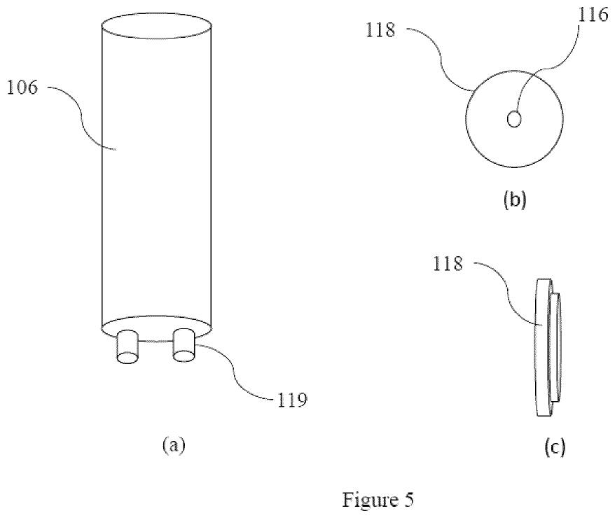

Example 7: Referring to FIG. 5, the tubular photobioreactor (106) as another example is shown. The tubular photobioreactor (106) has the following dimensions: the inner diameter is 5 cm, and the outer diameter is 5.5 cm. The height of photobioreactor (106) was 51.5 cm. The bottom of the photobioreactor (106) is connected to a foundational bottom that is slightly larger in diameter, measuring 6.5 cm. The reactor (106) is closed by a two-layered cap, as depicted in FIGS. 5(b) and 5(c). The upper layer of the cap has a diameter of 5 cm, while the lower layer has a diameter of 3.4 cm. Both layers are 0.3 cm thick. There is a 1 cm diameter hole (116) in the cap. Additionally, two circular outlets (119) are positioned at the bottom of the reactor (106). These outlets (119) have inner and outer diameters of 0.5 cm and 0.8 cm, respectively, with a 1.8 cm separation between them. The tubular photobioreactor (106) was affixed to a square platform of side of 15 cm by inserting the two outlets (119) into the two corresponding holes drilled in the platform. The height of the platform was 15 cm. Each reflector fin (108) had a height of 59 cm, a thickness of 0.3 cm, and a breadth of 5 cm. These reflector fins (108) were connected to the platform using a hinge-based mechanism. Each reflector fin (108) was positioned at an angle (α) of 90° relative to the base (102). There was a 15 cm gap between opposing reflector fins (108). The vertical gap between the adjacent reflector fins (108) was covered with adhesive aluminium reflectors (not shown in Figures) so that light enters into the photobioreactor system (100) only from the top. The setup was placed in the morning sunlight. A TSL2561 luminosity sensor connected to an Arduino Uno board that was in turn connected to an Arduino IDE in a laptop was used to measure the light intensity. Prior to installing the reflector fins (108), the light intensity near the bioreactor's base was in the range of 70,000 lux. However, after enclosing the area around the bioreactor (106) with the reflector fins (108) and using adhesive aluminium reflectors to enclose the gaps between the reflector fins (108), the light intensity near the bioreactor's base decreased significantly to approximately 5,000 lux. It is to be noted that, generally speaking, the intensity of sunlight as a function of the vertical distance of the enclosed space of the reflector fins (108) varies according to the time of the day.

The photobioreactor system (100) offers a highly adaptable and efficient platform for the optimal distribution of light and thereby the cultivation of photosynthetic organisms, making it suitable for various research and industrial applications. The photobioreactor system (100) is proposed to create an optimal environment for the growth of photosynthetic organisms, enhancing their productivity. The inclusion of reflector fins (108) positioned strategically in the system (100) ensures efficient utilization of light by reflecting it towards the culture of photosynthetic organisms. Different photosynthetic organisms have different light intensities for optimal growth. By adjusting variables like the tilt angle (α) of the reflector fins (108), the distance between the reflector fins (108) and the stack of bioreactors (104), etc, the light intensity can be modulated to our desired level. This promotes better growth and photosynthesis. The photobioreactor system (100) offers flexibility in terms of the positioning and adjustment of reflector fins (108), allowing for customization based on specific needs and experimental conditions. The base (102) of the system (100) is constructed for the positioning of a stack of photobioreactors (104), saving space and increasing the scalability of cultivation. As an example, one can vertically stack an increasing number of flat panel bioreactors (106) with an appropriate increase in the dimensions of the reflector fins (108). Similarly, one can increase the height of the tubular photobioreactors (106) or vertically stack rows of tubular photobioreactors with an appropriate increase in the dimensions of the reflector fins (108).

The stack of photobioreactors (104) are equipped with the culture circulation system that includes baffles (120) and impellers (122), ensuring the efficient circulation of essential nutrients to the photosynthetic organisms for their growth. The system's (100) dimensional range is versatile, accommodating various sizes and configurations to meet specific requirements, from small-scale experiments to large-scale industrial applications.

While preferred aspects and example configurations have been shown and described, it is to be understood that various further modifications and additional configurations will be apparent to those skilled in the art. It is intended that the specific embodiments and configurations herein disclosed are illustrative of the preferred nature of the invention and should not be interpreted as limitations on the scope of the invention.

The various embodiments of the present disclosure have been described above with reference to the accompanying drawings. The present disclosure is not limited to the illustrated embodiments; rather, these embodiments are intended to fully and completely disclose the subject matter of the disclosure to those skilled in this art. In the drawings, like numbers refer to like elements throughout. Thicknesses and dimensions of some components may be exaggerated for clarity.

Herein, the terms “attached”, “connected”, “interconnected”, “contacting”, “mounted”, “coupled” and the like can mean either direct or indirect attachment or contact between elements, unless stated otherwise.

Well-known functions or constructions may not be described in detail for brevity and/or clarity. As used herein the expression “and/or” includes any and all combinations of one or more of the associated listed items.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises”, “comprising”, “includes” and/or “including” when used in this specification, specify the presence of stated features, operations, elements, and/or components, but do not preclude the presence or addition of one or more other features, operations, elements, components, and/or groups thereof.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

### EQUIVALENTS

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

In addition, where features or aspects of the disclosure are described in terms of Markush groups, those skilled in the art will recognize that the disclosure is also thereby described in terms of any individual member or subgroup of members of the Markush group.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present.

Reference numerals:

| Reference Number | Description |
|---|---|
| 100 | Photobioreactor system |
| 102 | Base |
| 104 | Stack of photobioreactors |
| 105 | Columns |
| 106 | Photobioreactor (flat panel, tubular, etc.) |
| 108 | Reflector fin |
| 112 | First end |
| 114 | Second end |
| 116 | Hole |
| 118 | Circular lid |
| 119 | Outlets |
| 120 | Baffle |
| 122 | Impeller |

We claim:

1. A photobioreactor system (100) comprising:
a base (102);
at least one reflector fin (108) comprising a first end (112) and a second end (114), the first end (112) of the at least one reflector fin (108) is positioned adjacent to the base (102); and
a stack of photobioreactors (104) disposed on the base (102) to prepare culture of photosynthetic organisms;
wherein the at least one reflector fin (108) is configured to reflect light rays towards the culture of photosynthetic organisms, the at least one reflector fin (108) is defined with a vertical length at least equal to the elevation of the stack of photobioreactors (104).

2. The photobioreactor system (100) as claimed in claim 1, wherein the individual photobioreactors (106) comprising the stack of photobioreactors (104) is positioned one above the other.

3. The photobioreactor system (100) as claimed in claim 1, wherein the second end (114) of the at least one reflector fin (108) is adjustable with respect to the base (102).

4. The photobioreactor system (100) as claimed in claim 1, wherein the at least one reflector fin (108) is inclined at an angle (α) from the base (102).

5. The photobioreactor system (100) as claimed in claim 4, wherein the length of the at least one reflector fin (108) is such that even after adjusting for the angle (α) from the base (102), the at least one reflector fin (108) has a slant height at least equal to the elevation of the stack of photobioreactors (104).

6. The photobioreactor system (100) as claimed in claim 1, wherein a gap (G) separates two adjacent reflector fins (108).

7. The photobioreactor system (100) as claimed in claim 1 wherein the at least one reflector fin (108) is coated with a reflective material.

8. The photobioreactor system (100) as claimed in claim 6, wherein the gap (G) separating each of the reflector fins (108) from another is covered by a material.

9. The photobioreactor system (100) as claimed in claim 1, wherein the first end (112) of the at least one reflector fin (108) is positioned at a distance “x” from the base (102).

10. The photobioreactor system (100) as claimed in claim 1, wherein the stack of photobioreactors (104) comprises a culture circulation system having at least one baffle (120) and one impeller (122) configured to circulate essential nutrients to photosynthetic organisms.

11. The photobioreactor system (100) as claimed in claim 1, wherein the stack of photobioreactors (104) comprises at least one pump configured to circulate essential nutrients to photosynthetic organisms.

12. The photobioreactor system (100) as claimed in claim 1, wherein the photobioreactor system (100) comprises a translucent or an opaque cover configured to cover a top surface of the stack of photobioreactors (104).

13. The photobioreactor system (100) as claimed in claim 12, wherein the cover is positioned at a distance from the top surface of the stack of photobioreactors (104) and the cover defines a plurality of apertures configured to allow entry of light rays through the cover.
